(19) United States
(12) Patent Application Publication
(10) Pub. No.: US 2006/0233783 A1
(43) Pub. Date: Oct. 19, 2006

(54) Topical composition in the form of a gel for treating skin burns

(76) Inventor: Harold Armando Gomez Torres, Bogota (CO)

Correspondence Address: BIRCH STEWART KOLASCH & BIRCH, PO BOX 747, FALLS CHURCH, VA, 22040-0747, US

(21) Appl. No.: 10/552,011

(22) PCT Filed: Apr. 9, 2003
(86) PCT No.: PCT/IB03/01295

§ 371 Date: Oct. 7, 2005

(51) Int. Cl. A61K 38/46; A61K 9/14; A61K 31/24

(52) U.S. Cl. 424/94.65; 424/486; 514/537

(57) ABSTRACT

The present invention relates to novel topical compositions for treating burns, grazes, erythema, eczema, herpes infection, evulsion, sores and any skin damage leading to sloughing, including at least three components, namely a first barrier gel, a second barrier gel and an active principle having proteolytic activity. The novel compositions have enhanced pain-killing, protective, débrding and anti-inflammatory effects relative to the conventional products used in the art.

### FIELD OF THE INVENTION

The present invention relates to novel topical compositions for the local treatment of burns, grazes, erythema, eccema, herpes infection, evulsion surface sores and any skin damage leading to gangrene and in particular, a composition creating a translucid colloidal film on the injury covering the nervous terminals (pain relief), reducing nervous irritability, isolating from the external media preventing from contact with noxious substances, maintaining dryness of injury and doing pressure (dressing effect) for creating a media permitting effective and fast cell re-generation; while enzymatic action causes des-inflammation, debriding and cleaning the zone.

### BACKGROUND OF THE INVENTION

Medical and Clinical Environment

Traumatic injuries of skin, i.e., burns, scald, abrasion, evulsion, etc., have been treated by plastic surgery, addressed to the theme, with scientific perspective and related researches.

Reconstructive surgery of burned people, applied science forming part of the plastic surgery specialization, is an area wherein a specialized physician works re-constructing tissues, treating burns and repairing lost skin.

In the case of surface skin injuries and burns, although its physiopathology is clearly known, even beginning twenty one century there is no a consensus in treatment of same showing deep ignorance in this issue and a big part of physicians guides in an empiric form or by very basic information.

This situation has caused that in this field of medicine an enormous number of products of different origin be applied, sold or indicate as a treatment, from empiric substances, plants, coffee, white of an egg, Aloe vera, mucilage, etc., until tannins, mercury and topic antibiotics are the king of substances used for treating skin injuries for burns (or abrasion) demonstrating lack of only one view.

Antibiotic and healing methods of therapy have a popular focus with variety of substances being more notorious sulfas, furazolidone, tetracycline, gentamicyne, mercury-chrome, epithelial growing factor and tannins, with studied and known results. However, local treatment of principal symptoms (pain, inflammation, debriding effect) has no received important pharmacological attention.

Antibiotic substances as silver sulfadiazine, furacine (fucidin), terramicyn and other of different type have treated to occupy this space of medical therapeutic. Silver sulfadiazine is more successful and has bigger market. However, scientifically speaking, it is an imperfect product to manage non-infected skin injuries.

The basic concept considers that this injuries healed by itself (epithelialize), with no importance of the substance used, whenever complications do not arise.

Philosophy is producing comfort to the patient while organism generates cicatrisation process by itself.

Burns and Evulsions

Burn is defined as a skin injury produced by energy transfer, from a thermo source to the body, highly enough to cause injury, possibly by direct transmission (caloric), chemic injury or electromagnetic radiation.

Immediate clinic manifestations in burn are changes in color from erythema to necrosis, deep pain in surface cases and presence of organic liquids by transweated.

Burn arises when cells are destroyed by heat, liberating chemical substances stimulating nerves causing pain, generating skin continuity loss with underlying elements exposition and, depending on the deep level, liquid loss by evaporation.

Burn healing mechanism is similar to that of a wound or abrasion, in second degree serum pimples are formed acting as a protective cover forming a new skin layer under them from the burn boundaries.

If burn is big and exposed is easier for bacteria to enter.

As a consequence many factors come into play as the continuity skin loss, necrosis (dead) of the skin sector affected, deep pain, hydro-electrolitic response of the organism, inflammation by liquids and chemicals, blushing by vasodilatation and further possibility of bacterial colonization.

In the same way defense mechanisms against hot is bringing into play, abundant sweating to bring down temperature by evaporation with liquid loss, hot dissipation by vasodilation and tissue resistance to hot or radiation (principally muscles and skin, nerves and vessels are very sensitive). It is considered that until 440 no cell injury arises unless of a prolonged exposition.

Epidemiology

Burn is one of the more frequent injuries occurring to human beings. In the United States about 3.5 to 4 million people visit physicians for diagnosis and treatment of burns.

Burns occupy a big percentage of medical consultation in hospitals and consulting rooms, 8 out of 10 persons has some type of burn during a year, being 95% of burns of home or ambulatory manage.

In the moment of a burn dead cell occurs, an event series similar to wounds begin:

1—Inflammation: is the acute reaction of tissues after injury, immediate response is vasoconstriction by nervous stimulus and thrombosis.

2—|Follows a vasodilatation and increase in the capillary permeability in the next 12 to 48 hours, according to the injury level, with leaving of blood fluid containing proteins, electrolytes and water.

Principal protein is albumin giving the plasma oncotic pressure (liquid retention) and passing to the extra-vascular space in the burn retaining liquids in which is called as edema.

In cell migration, by the increase in capillary permeability, specialized cells in responding to injuries arrive: leucocytes (macrophages and neutrophils (circulation immune white cells) in charged of cleaning and disinfecting this area, defense system against bacteria and dead cell elimination).

In respect to the chemical substances of dead cells, plasma and neutrophils some chemical substances are produced: euglobine, (capillary permeability), catecholamine, leucotaxine, bradykinin, calidine, kallicrein, histamine, serotonin and prostaglandins, all substances causing nervous stimulation, immune cell activity, vasodilatation, cell migration (chemotaxis) and other inflammation related changes.

Burn Classification

It is important to know burn classification according to coetaneous depth, etiology and extension.

Burns are classified according to diagnosis, treatment and prognosis parameters.

a) Depth

It is divided into three categories:

First Degree:

First degree—Superficial: only outer layers of epidermis or cornea layer are affected. It is characterized by an erythema of red color, deep pain, local heat, contact air sensitivity and spontaneous healing in three to four days. It could produce skin hyper-pigmentation. An example is sun burn: healing occurs in few days without scar.

Second Degree:

Superficial: partial or complete epidermis injury but with intact epidermis annex or indentation, deep pain, erythema, phlyctene, fast capillary filling, soft yet skin. An example is scald healing in 8 days.

Deep: epidermis complete destruction (included germinative stratum) and part of dermis, flyctenes, pale rose tone, moderate pain (due to nervous destruction), hard and cardboard-like skin, slow capillary filling and delay healing beginning in the annexes (hairs and glands), and almost always scare is left. An example is steam or flame, in which case regeneration occurs in 16 days.

Third Degree:

There is a total compromise of skin, there is not cell regeneration, white, insensible, cardboard-like, dry skin without edemas and can involve organs different than skin, as for example, electric, chemical and fire burns.

This burns always needed specialized medical attention.

First and second degree surface burns have spontaneously healing and are the principal object and applicability of composition of the present invention.

Etiology

Determinate origin of burn is always important to define lesion intensity, treatment and prognosis.

Sun, biological, steam, flame and scalds burns cause more surface burns, direct fire and chemicals burns cause middle burns and contact burns, deflagration and electric burns are the most dangerous.

### Consideration and Disposition of Burns

Extensive Burns:

These are burns involving more than 25% of an adult or 10% in children and with more than second degree depth. Apart from local injuries as necrosis, pain, vasculitis, edema, transweating and over-infection, there is systemic implication in which immunological reactions, vasodilatation, liquid emergence to the interstitial space, protein loss, necrosis residues, general sepsis and implication of vascular and urinary systems are presented. In these cases patients manage is exclusively made by physicians and in hospitals with liquid, proteins and electrolytes reposition, wound hospital and affected systems care (airborne ways) and in depth cases surgical treatments with grafts, flaps and reconstructive chirurgical processes. These are slowly healing patients and can be much time in the hospital. There are hypertrophic scares, deformations and hair loss sequelae. Patients having inhaled smoke are of special care for production of airborne ways illness, insufficiency and dead. Manage with antibiotics is indispensable both for the wound and in general because all patient with extensive burn suffers of over-infection.

Little, Minor and Superficial Burns.

It is considered a superficial burn those which can be ambulatory treated in house or in doctor's office without complications and not surpassing 25% sct and of second degree in adults, and 10% and second degree in children.

According to the parameters established, these are burns with no electrolytic implication of the organism, immunologic and vascular implication is little, and no infection is presented with exception of over-aggregated situations.

In this cases treatment is focus in preventing an over-infection, liquid loss, des-inflammation of the zone, offering comfort, offering analgesia, cleaning the zone, covering the burn area and protect it from the environment while the intrinsic healing processes act.

If a burn is little, it is not depth and it is not complicated, treatment consist of covering the zone, cleaning it, inspection it, washing it, take away the pain and debriding it; preventing over-infection and permitting re-epithelialization and complete healing in a maximum period of 3 to 5 days; analgesic, antibiotic and other local covering products are avoided. This local treatment is the object of the composition of the present invention.

Objective of Burn Treatment

Objective of local burn treatment is protect against infection and trauma, diminishing the pain, des-inflammation and accelerate removing of dead tissue while promoting methods enhancing scaring. Superficial burns epithelializing faster doing it with less scar.

Nowadays, most common methodology for treating superficial burns includes generally use of topic antimicrobial agents. Preferably silver sulfadiazine (SSD). This drug was developed in the 60's and is effective for controlling antimicrobial growth in burn while the eschar separate. SSD has a hydrophobic molecule making that the application of cream induces accumulation of significant amounts of proteinaceous exudates in wound surface.

These exudates are called PSEUDOESCHAR. Efforts should be carried out to take away this pseudoeschar that is a strong layer of material in burn surface, or in the contrary, paradoxically, bacterial colonization can progress. Therefore use of SSD in burns requires surveillance and periodical chirurgical debriding for removing eschar and the accumulated proteinaceous necrotic residues.

The epithelization process requires the burned zone clean and free of debris, requiring in the case of SSD the removal of necrotic tissue that unfortunately can be extremely painful and stressing for the patient, and further requires the use of great dose of analgesic.

The endogene proteases are produced by various cells in a burned zone. These enzymes enhance the liquefaction and removal of the necrotic tissue; the devitalized proteic residues must be removed in order to allow the epithelial cells to migrate and repair the burned zone. The collagenases proteases (enzymes) of intrinsic production that act exclusively on the collagen to denature it and making it more easy to be degraded by less specific proteases.

During decades exogenous proteases preparations have been made to accelerate the debriding process of the burns and wounds increasing the local proteic degradation rate and thus accelerating the epithelization process. This turns into decreasing the intensity of the injury or wound, less hours for taking care of the wound, and less malaise of the patient. The exogenous collagenase can be obtained in an enzymatic preparation derived from the clostridium histolyticum bacteria.

Pain and Trauma Over the Burn or Surface Abrasion

During the 12^{th} annual congress of the Wound Handling European Association in Granada, Spain between may 23 to 25, 2002, the attendants concluded that for the prevention of the ill treatment or trauma on a wound (healing) and pain preventing to the patient, the most important elements related to the care of the wound should be taken into account. The removal of the dressing is a big cause of pain and therefore obtaining a dressing that eliminates or diminishes pain and trauma is a highly desired characteristic.

Proteolytic Enzyme Function in the Burn Repair

The wounds of all kinds, including burns, possess a common fact: they all produce a physiologic response. The severity of such response varies with the degree and type of wound.

The hyperemia is a physiologic response to trauma, which is followed by flare, that is a previous requirement to healing and then causing an edema, which usually delay curing. If the edema is excessive, it can delay the tissue metabolism increasing the possibility for infection, ischemia and hypertrophic scars. It is therefore convenient to use a method that reduces the edema.

The edema represents a liquid excess and cell remainder within the tissue gaps and its elimination depends on the liquid drainage (for example, by applying pressure) and on the proteolysis, that is the increase of the removal of the proteic remainder by proteolytic enzymes. It has been proved (Medical Tribune 354 1968) the enzymes from the carica papaya reduce to a minimum the edema associated with flare in the wounds being healed. Such fact correlates directly with a significant decrease or absence of pain.

State of the Art Products for Burn Treatment

Starting with empiric substances, herbs, Aloe vera, mucilage etc., and continuing with tanines, mercury, and topic antibiotics are the wide range of used substances to treat skin wounds caused by burning (abrasion) which simply demonstrates the lack of unity in criteria to that respect.

Empiric treatments with coffee, onion, white of egg and other different substances with traditional knowledge are used in addition to a medical handling based on antibiotics and crust forming substances such as chromium mercury associated with analgesics and lubricants for the mentioned wounds.

Many other different products have been used with average results, such as cerium nitrate, Iodine (Causes pain), tannins, rifampycin, and triconjugate treatment consisting on silver nitrate plus chromium mercury plus tannic acid. This treatment has an antiseptic weakness and produces a scab that can predispose to bacteria culture.

The practice of topic antibiotic therapy for burns was not designed to treat the recent surface wounds, which handling management is quite different. The local antibiotic therapy must be kept for those clinic cases in which the burn sepsis due to its magnitude can turn into a major problem. The patient with a recent surface burn will not benefit by using antibiotics.

Some of the Products are

Mafenide: (sulfamilon) which is a methylated sulfonamide (sulfa group) effective against a wide bacteria group, in particular the clostridium, which can penetrate the scab and cause a metabolic acidosis.

Silver nitrate: An inorganic salt having a poor wound penetration, helps removing the scab, under bacterial spectrum.

Silver Sulfadiazine: Comprises sulfadiazine and silver nitrate, penetrates the scab and is effective against the entire burns bacterial spectrum.

Gentamycin: Used against the pseudomona aeruginosa, possesses a quick bacterial resistance.

Nitrofurazones: They have a reduced bacterial spectrum.

Others: The butesyn Picrate, methatinate, aloe vera, epidermis growth factor (Cuban product) and other substances without therapeutic significance are found in the market.

Use of proteolytic enzymes: The application of proteolytic enzymes on a burn wound with local sepsis has a big importance as it disrupts the coagulation, eliminates the accumulated proteinaceous material that “covers” the bacteria with the antibiotic action and thus increases the antibiotic effectiveness preventing the infection.

### DESCRIPTION OF THE INVENTION

The present invention provides a topic composition for treating burns and coetaneous injuries causing sphacelus, from every one of the factors originating the burn or surface abrasion: pain, for which the thickener substance was designed similar to a second skin (that is why it causes analgesia); flare, for which the proteolytic enzyme was designed having an enzymatic debriding effect, being those the basic concepts of gel.

Another objective of the present invention is to provide a composition that besides the above-mentioned components, it also can contain other components effective on secondary factors of the burns, such as including antiseptic (chlorexidine) in case an infection is suspected, urea for a better lubrication and anesthetic (idocaine) for the painful wounds in adults and in particular in children.

The sepsis of the burned injury or burn is defined by Teplitz as: Presence of bacterial organisms exceeding 100,000 colonies per tissue gram in the burned tissue and that are invading the tissue under the burned zone (artz Chap. 17, Pg. 250)

During a short period of time after the occurrence of a burn, the wound remains sterile up to an average of 48 hours, the later contamination comes from the external medium, from the surrounding skin (Saprophilous) and other sources such as respiratory and feces. It is important to recognize that the topical antibiotic therapy has been designed to control the sepsis of the burn and not for the routinary treatment of little burns in which the sepsis is not the problem.

Having clearly understood the concept of sepsis of a burned wound and its possibility of appearance or not in the burn's initial phase, the use of an adequate therapy is rationalized. An overutilization of topical antibiotics can produce the opposite of the desired effect (overtreatment) due to the saprophilous bacterial proliferation.

A few hours after the burn, microbiologically a surface bacterial colonization is initiated with a great variety of organisms, in particular positive gram coccu (mainly the staphylococcu). This colonization is started by the hair follicle and perifollicular tissue. After a period of 3 to 5 days the negative gram organisms are predominant which initiate the invasion of the burn underlying tissue. Dissemination through the lymphatic paths to the blood stream takes place. There are some factors that bias the bacterial over-infection such as the vascular destruction inhibiting the nutrients apportion to immune cells, the necrosis of coagulation that increases with the over-infection and the vascular necrosis. It has been widely proved that burns inhibit the immune response (vascular necrosis).

The topical antibiotic therapy does not sterilize the burn, just and simply reduces the number of bacteria intending to allow the immunological mechanisms of the host to control the infection.

As the burn flower is not absolutely eradicated, the effort is addressed to allow the replacement of the coetaneous cover.

When there is a bacterial colonization, this is initiated on the surface where there is dead tissue and deeps in progressively. Having wider affected area, wound deepness and longer time of occurrence, the greater the possibility of infection. The age, nutritional and immunological condition of the individual, being exposed to the surrounding environment, persistent flare, wound location and wound detritus are important factors. A minor burn without any scab (detritus), clean tissues and isolated from the environment and unflare, presents the best defense against over-infection. It is imperative to know that a topical antibiotic therapy on a burn is directly addressed to control the appearance of the sepsis on the burn and not as a routine treatment of small burns in which the infection is not a threat or a problem.

Today there is a novel complementary approach different from the local therapeutics of burns, named HYDROGELS, directed to offer comfort, analgesia and pain relief in short time in the burned area, besides an antiflare and debriding effect. Such approach is not an antibiotic therapy, nor has been formulated for scab removal, the deal to form a smooth, transparent and colloidal layer that isolates the area and thus, prevents the bacterial over-infection.

Under the above concept, the new composition of the present invention was designed from each one of the factors originated by the burn or surface abrasion: pain, for which the thickener substance was designed similar to a second skin (that is why it causes analgesia); flare, for which the proteolytic enzyme was designed having an enzymatic debriding effect, being those the basic concepts of gel.

One can also add new components for the secondary factors of the burns, such as adding chlorexidine in case an infection is suspected, urea for a better lubrication and anesthetic (idocaine) for the painful wounds in adults and in particular in children.

The indication of the present invention are for first grade wounds, second grade superficial wounds, not infected, not being located in special areas and that have less than 25% of extension.

The composition of the present invention has a new clinic focus with the following characteristics: forms a transparent film, antifraring, pain relief, isolates the wounded zone, has a rheological power, prevent infection, is water absorbent and produces a fast and efficient epithelization.

The composition is a viscous transparent gel contained in a plastic tube designed to be applied and spreaded directly on the affected area. Is a new physiological view in topical treatment, symptomatic and preventive in the pathology of superficial and non-infected burns or local avulsions.

International articles refer to the debriding and antiflaring effect of the papain, which in addition of the barrier effect or second skin is used in the product.

In the design of the composition of the present invention, the mix, affinities and properties of the described substances focused on the pathology for which they were prepared, results in a specific formula adequate for treating signs and symptoms that show in burns or avulsions.

This new composition offers comfort when used and in its application, mediate or immediate analgesia and a proteolytic debriding effect. Form a transparent layer that allow a direct view of the wound and has an apposite colloidal effect that exerts pressure isolating immediately from the surrounding environment.

The decrease of liquid loss, the easy handling and the mobility of the affected zone addressed to an effective prevention of over-infections and to a fast growth of the tissue. The composition also offers other advantages such as easy application and removal, being free of adverse effect for the patient, is no toxic to the tissue, does not produce pain when applied according to the indications, has an immediate analgesic effect, does not stain or bleach the wound and has low cost.

Mechanism of Action

The composition creates a transparent colloidal film over the wounded zone covering the nervous terminals (pain relief), isolating from the external environment in order to prevent contacting harmful substances, maintaining a dried zone and applying pressure (apposite effect) to create a medium allowing a fast and reliable cell regeneration; while the enzymatic action reduces the inflammation, debrides and cleans the zone.

The market of the available products for handling burns and superficial abrasions is somewhat uncertain, as they are substances that were not designed to follow the physiopathology course of the wounds and they just refresh and act as topical antibiotic or give temporary relief without being specific in pain relief and antiflare.

The basic concept of the composition of the present invention is that of treating with each one of their components all the aspects of the physiopathology of burns; the pain happens due to the nervous terminal exposition and the gel of the invention creates an external transparent layer that covers the skin while the natural and normal epithelization process takes place. Said layer help that process to develop faster as it makes the medium and conditions more adequate (cleans, debrides, protects).

The inflammation occurs due to the injury reacting physiological processes (vasodilatation, cell migration, active substances liberation such as histamine and serotonine) and the efficiency of the papain and enzymes are proven to act well in the topical treatment and handling of the dermic inflammatory processes. Therefore, it was found that the combination of barrier enzymatic protecting substances looking for a new management in the burns and superficial abrasions treatment was ideal to said treatment.

Components of the Composition

a. The papain. Is a plant proteolytic enzyme extracted from the Carica papaya that hydrolyses peptidic, amidic and steric bonds of the proteins.

Its properties are having a good proteolytic activity, good thermo stability, are thermo soluble, anti-inflamatory and have a debriding effect. In particular, has a proteolytic activity between pH 3 and 9, a wide range of thermo stability (up to 70° C.), is poor in germ s content and dissolves easily in water, and has a high effectivity in viscous solutions.

The papain has many applications and uses: is a digestive substance that promotes or substitutes other digestive enzymes is antihelmintic destroying the proteic cuticle of intestinal worms, leather, tobacco, textiles and meat smoother industries. IN wounds and burns it presents a proteolytic activity on dead tissue, without affecting the live tissue, causing an enzymatic scrubbing and an optimal healing. It has an inherent anti-inflamatory effect and is able to be combined with certain antibiotics.

It is also used in biochemistry in breaking the bonds and to determine chemical structures of other proteins (as in the determination of human Ig G).

The papain is a protease that catalyze the hydrolysis of esters and peptides. The most important amino acids comprised in it are: triptophan, tyrosine, phenyl-alanine, histidine and arginine.

The papain is used preferably in the composition of the present invention in the range of 0.2 and 5% by weight of the composition, preferably in an amount of around 0.5% by weight of the composition.

b. The carboxymethyl cellulose. This component is a synthetic resin derived from the acrylic acid, is a thickener, emulsifier and interface coalescent (consistence). Its properties in the composition of the present invention are:

Protecting barrier, or second skin that isolates the wound while the papain acts.

Gives the necessary stability, film producing agent and physiologically inert.

Good antibacterial barrier.

This component is a well known product and is used in various field of industrial production such as: food, textiles, detergents, cosmetics, paints, adhesives, ceramics, toothpaste, leather, etc. This is a cellulose derived anionic polymer and hold the following properties:
a. Dissolves very easy in cold or hot water.
b. Acts as a thickening agent, suspension agent and suspension stabilizer.
c. Hold in the water thus contributing with the dryness of the underlying wound.
d. Acts as a film producing agent that is oil, fat and organic solvents resistant.
e. Acts as binding and as colloid protector.
f. Is a rheological control agent.
g. It is physiologically inert, an essential property for the searched effect.

The CMC solution does not turn solid with heating, it only diminishes its viscosity when the temperature increases above 40° C., has a high resistance to microbiologic attacks and when subjected to long term storing the recommendation is use of preservatives to avoid the decrease in viscosity and its degradation. It has also stability within a wide range from pH 4 to pH 9 being the preferred pH neutral.

The prefer range of use of this component is between 1.0 to 4% by weight of carboxymethylcellulose gel and this gel is present in a range of 71.5 to 77.5% by weight of the composition of the present invention.

c. CARBOPOL. This a synthetic resin with a high molecular weight, polymerized with a hydrophobic monomer, obtaining a polymer with crosslinked chains extracted from the acrylic or polyacrylic acid. His chemical name is carboxypolymethylene.

It is mainly used as thickener and emulsifier, its function is maintaining the homogenization of the preparations stabilizing emulsified systems against sedimentation or separation, absorbing the respective interface (oil-water). The CARBOPOL coalesce rapidly the application of the product giving it consistence when stabilizing and thickening the emulsions.

Its advantages are:
a. it forms a barrier that protects the skin from new potential external irritants.
b. it cleans nastiness and removes the undesired oily substances.
c. it uniformly distributes the preparation on the skin.
d. it accelerates the stabilization of preparation.
e. its stability for two years at room temperature.
f. low concentrations of CARBOPOL are needed to get the desired effect.
g. it eliminates the need of emulsifier soaps.
h. it is translucent and does not produce any coetaneous irritation.
i. if occasionally contacts the eyes, it can cause minor irritation.
j. not poison when ingested.

There are many types of carbopols, the most important are Carbopol 941, Carbopol 940, Carbopol 934, Carbopol ultrez 10, Carbopol etd-2020. Carbomer polymers have been used for rheological control (structure constructive agents) in lotions, creams and gels. Polymer molecules have a unique ability to increase the thickness of liquids in which they are dissolved (dispersed), including very wet-concentrations. This is because of the voluminous expansion capacity (water absorption) of carbomer microgels.

Polymer capacity to increase the thickness depends on its “intrinsic viscosity”. “Intrinsic viscosity” is expressed in dL/g. Factors that affect intrinsic viscosity of carbomer polymer are: pH, types of electrolytes, ions concentration.

Microgel particles in polymers increase the thickness of a solution by means of two mechanisms: 1) increasing viscosity according to the polymer swelling, and 2) increasing viscosity by microgel stiffness.

The preferred range for use this component in the composition is between 1.5% and 2.5% by weight of Carbopol gel, and the amount of Carbopol gel is present between 22-28% by weight of composition.

Optionally, the composition comprising the three components a., b. and c. above described may also include an analgesic with the aim to bock the nervous conduction, when they are locally administered. Lidocaine is the most stable local anaesthetic, and therefore, the most used nowadays. It is used in local anaesthetic solutions and for mucous, and also as injectable anaesthetic, infiltration anaesthesia, and in cardiology as a modifier of cardiac rhythm. It is used in a composition range from 1% to 5% by weight of the composition.

### EXAMPLES OF COMPOSITIONS FOR DIFFERENT TYPES OF APPLICATIONS

### Example 1

In a first embodiment, the composition of the present invention is prepared in three steps:

a) First, a CARBOPOL gel is prepared which is present in a composition in 25% by weight.

b) Secondly, a carboxy methylcellulose gel is prepared which is present in the composition in 74.5% by weight.

c) Finally, 0.5% by weight of papain is added to the composition.

a. CARBOPOL GEL. This gel is prepared according to the next composition:

2.00% Carbopol,

2.23% Triethanolamine,

95.77% Distilled Water.

Total amount of CARPOBOL gel 100.00%.

b. CARBOXIMETHYLCELLULOSE GEL. This gel is prepared according to the next composition:

3.00% Carboxymethylcellulose Sodium

0.50% Propyl Parebene,

0.50% Methyl Parabene,

96.00% Distilled Water.

Total amount of carboxymethylcellulose gel, 100.00%.

c. ACTIVE PRINCIPLE. PAPAIN

0.50% PAPAIN.

Formula of standardized batch for manufacturing: 5,000 g

RAW MATERIAL AMOUNT

PAPAIN 25 grams,

CARBOPOL GEL 1,250 grams,

CARBOXYMETHYLCELLULOSE GEL SODIUM 3,725 grams.

TOTAL AMOUNT RAW MATERIALS 5,000 grams.

According to the above established percentages, next are the necessary amounts for manufacturing the composition subject of the present invention:

a. CARBOPOL GEL: 1,250 g

RAW MATERIAL AMOUNT

Carbopol 25.0 grams,

Triethanolamine 28.0 grams,

Distilled water 1,198.0 grams

Total Raw Materials 1,250 grams

b. CARBOXYMETHYLCELLULOSE SODIUM GEL: 3,725 grams.

Carboxymethylcellulose Sodium 112.0 grams,

Propyl Parabene 19.0 grams

Methyl Parabene 19.0 grams,

Distilled Water 3,576.0 grams

c. PAPAIN 25 grams

2. Example of the manufacturing process:

a. CARBOPOL GEL

1. Take a 2 kg stainless steel capacity container.

2. Pour the distilled water in the stainless steel container.

3. Slowly add the triethanolamine into the container.

4. Start the stirring process with a stainless steel shaker.

5. Keep on stirring while slowly the Carpobol is added.

6. Pour into the mixer, stirring at minimum speed for about 15 min until dissolution is complete and a transparent gel is obtained.

b. CARBOXYMETHYLCELLULOSE

1. Take a 5 kg stainless steel capacity container.

2. Pour the distilled water in the stainless steel container.

3. Slowly add the carboxymethylcellulose into the container.

4. Start the stirring process with a stainless steel shaker.

5. Keep on stirring while slowly adding the propyl parabene.

6. Keep on stirring while adding the methyl parabene.

7. Warm the mixture at 50 to 60° C., constantly stirring.

8. Stop heating and keep stirring until the mixture reaches room temperature.

9. Pour into the mixer, stirring at minimum speed until the mixture reaches 17° C.

c. PAPAIN

1. In the stainless steel container pour the CARBOPOL GEL.

2. Slowly add the CARBOXYMETHYLCELLULOSE GEL into the container.

3. Start the stirring process with a stainless steel shaker.

4. Keep on stirring while slowly the PAPAIN is added.

### Example 2

In a second embodiment, a composition having the next components is provided:

a. First substance: it is a proteolytic enzyme, particularly papain derived from Carica papaya, which healing and anti-inflammatory characteristics are used for the treatment of wounds.

b. Second substance: CARBOPOL.

c. Third substance: carboxymethylcellulose sodium salt.

d. Forth substance: local anaesthetic drug.

The composition or quantitative formula from the product is prepared in three steps, according to the next description:

1. 25% CARBOPOL GEL

2. 72.5% CARBOXYMETHYLCELLULOSE GEL 2.2.0% LIDOCAINE

3. 0.5% PAPAIN.

The composition of the present invention is prepared in three steps:

a) First, CARBOPOL gel present in the composition in 25% by weight is prepared.

b) Then, carboxymethylcellulose gel present in the composition in 72.5% by weight is prepared.

c) Finally, 0.5% and 2% by weight, based on the total weight of the composition, of papain and Lidocaine, respectively, are added.

a. CARBOPOL GEL. This gel is prepared according to the next composition:

Carbopol 2.00%,

Triethanolamine 2.23%,

Distilled Water 95.77%.

Total amount of CARBOPOL gel 100.00%

b. CARBOXYMETHYLCELLULOSE GEL. This gel is prepared according to the next composition:

Carboxymethylcellulose Sodium 3.00%,

Propyl Parabene 0.50%,

Methyl Parabene 0.50%,

Distilled Water 96.00%.

Total carboxymethylcellulose gel 100.00%

c. ACTIVE PRINCIPLE. PAPAIN

Papain 0.50%.

d. ANAESTHETIC.

Lidocaine 2.00%.

2. Example of the manufacturing process:

a. CARBOPOL GEL.

1. Take a 2 kg stainless steel capacity container.

2. Pour the distilled water in the stainless steel container.

3. Slowly add the triethanolamine into the container.

4. Start the stirring process with a stainless steel shaker.

5. Keep on stirring while slowly the Carbopol is added.

6. Pour into the mixer, stirring at minimum speed for about 15 min until dissolution is complete and a transparent gel is obtained.

b. CARBOXIMETILCELULOSA GEL

1. Take a 5 kg stainless steel capacity container.

2. Pour the distilled water in the stainless steel container.

3. Slowly add the carboxymethylcellulose into the container.

4. Start the stirring process with a stainless steel shaker.

5. Keep on stirring while slowly the propyl parabene is added.

6. Keep on stirring while the methyl parabene is added.

7. Warm the mixture at 50 to 60° C., constantly stirring.

8. Stop heating and keep stirring until the mixture reaches room temperature.

9. Pour into the mixer, stirring at minimum speed until the mixture reaches a temperature of 17° C.

c. PAPAIN AND LIDOCAINE

1. Pour the carbopol gel into the stainless steel container.

2. Slowly add the carboxymethylcellulose gel into the container.

3. Start the stirring process with a stainless steel shaker.

4. Keep on stirring while papain and lidocaine are slowly added.

Preparation of the composition of the present invention with chlorhexidine and urea is similar to the above and follows the same parameters as the procedure above described.

### Example 3

Clinic Results are Comparative with the Products Already Existing.

Clinical evaluation of the product was made, where datum of the patient, a brief anamnesis, a description of the wound and time monitoring picture with the variables PAIN, INFLAMMATION and HEALING EFFECT.

Furthermore, the presence of overinfections was investigated, and the result was negative.

STUDY GROUP: 44 Patients diagnosed with burns or avulsion that fulfil the requirements to apply the composition of the present invention.

Administration Scheme, Doses, Route and Frequency

The product under study is exclusively for coetaneous application, and once the wound has occurred, application of topic doses is distributed each 2 hours, modifiable according to the process of skin renovation.

Comparative study of the composition of the present invention was made with aloe vera (substance derived from sabila, recommended and publicized for handling burns and similar composition to the present application), both in gel packaging.

None antibiotic cream was used in this study, since infected wounds or areas where the process of bacterial growing has occurred are not the objective.

Most of the wounds treated fluctuated in an extension between 1 to 10%, excluding some patients that received the present composition in spread out burns up to 30%. All the wounds were of first and second grade according to the depth, capable to improve with these products.

Not important complications were observed, and some burns treated with Aloe Vera continued the normal infectious process that is common in these cases. Products were applied according to the next evaluation times:

0 Hours: Initial clinical evaluation.

6 Hours: during this period of time, symptoms of these specific wounds are stronger.

24 Hours: during this period of time, symptomatology of all wound caused by burns of first and second grade in small areas, finds stability starting its resolution during the natural process.

72 Hours: natural development of this kind of wounds is in the recovery sep, with the absence of most of the symptoms and signs.

Performance Analysis with Aloe Vera:

As a helper in the initial symptomatology, it freshens, calms and, as a part of the general measures, it has some level of efficiency without being the ideal product in reference to the evolution thereof.

In general, patients believe that the product “freshens, is good” and helps the initial comfort of the wound, meanwhile in subsequent hour, it does not have any Clinical incidence, all related with natural evolution of the wound, its extension, depth and localization. 50% of the patients consider the product is good, between good and excellent 10%, and regular 12%.

In general, medical concepts are good, 52%, patient comfort improves, excellent 10% and, inflammation remains the same, 30%. Most of medical reports declare persistence of discomforts related to pain and inflammation, and aqueous characteristic of the Aloe.

Evolution of Pain:

Most of the patients had agonizing pain at the time of the initial evaluation.

6 hours before Aloe's application, intensity of pain was of less intense, although some patients still had intense pain (13%.) 24 hours later: some patients still report between moderate and minor pain and, but 70% without pain.

72 hours later: 5% of the patients with moderated pain, 18% minor and 77% without pain.

Evolution of the Inflammation:

Most of the wounds were small.

6 hours later: A patient has severe inflammation and 33% have minor inflammation.

24 hours later: 30% of the group still have minor inflammation and, almost 50% moderated.

72 hours later, 36% of the patients still reports minor inflammation.

Evolution of the Cleaning:

Not significant.

Analysis of Results with the Composition of the Example 1:

The concept emitted by the patients with respect to the product is in superlative and excellent grade in 48%, good 42%, 10% of patients do not emit a concept, there are not regular concepts. In some cases, its reported minor annoyances at the time of the application, and a fast relief of the pain during the whole study. The epithelialization and remove the inflammation occur in a short period of time.

Medical concepts are equally in superlative grade, very good and excellent 325, and good 46%; magnificent analgesia, efficient product, easy to handle and use in wide and serious area

Evolution of the Pain with the Composition of Example 1:

6 hours later: 35% of the patients have intense pain at the hour zero, and six hours later, this percentage diminish to 3%.

24 hours later: pain is minor and, 87% do not have pain.

72 hours later: Only 3% of the patients have a minor grade of pain and, 89% do not report pain.

Evolution of the Inflammation with the Composition of Example 1:

6 hours later: one patient with intense inflammation, 35% with minor inflammation and, 46% without inflammation.

24 hours later: Only one patient reports intense inflammation, most of them (78%) do not have inflammation.

72 hours later: 2% report moderated inflammation and, 85% do not have inflammation.

These results confirm effectiveness of the product on pain an inflammation. As it can be seen, compositions of the present invention have superior analgesic, protective, healing, and anti-inflammatory effects in reference to all of the previously known in the state of the art.

The above examples should not be construed as limiting of the scope of the present invention and the scope of the same is determined by the claims appended hereto.

1. A topic composition in form of a gel for treating skin burns comprising:
A first barrier gel for skin protection comprising an thickener agent and an emulsifier agent in an aqueous carrier;
A second barrier gel for skin protection comprising a second thickener agent, a preservative agent, in an aqueous carrier; and
An active principle having proteolytic activity.

2. A composition according to claim 1 wherein the active principle is papain.

3. A composition according to claim 1 furthermore comprising an anaesthetic agent.

4. A composition according to claim 3 wherein said anaesthetic agent is lidocaine and is present in a range between 1 to 5% by weight of composition.

5. A composition according to claim 1 wherein said first thickener agent consists of carboxypolymethylene in an amount between 1.5 and 2.5% by weight of said first gel.

6. A composition according to claim 1 wherein said second thickener agent is carboxymethylcellulose in an amount between 1% and 4% by weight of said second gel.

7. A composition according to claim 1 wherein the first gel is present in an amount between 22% and 28% by weight of the composition, said second gel is present in an amount between 71.5% and 77.5% by weight of the composition and the active principle is present in an amount between 0.2% and 5% by weight of the composition.

8. A composition according to claim 3 wherein the first gel is present in an amount between 22% and 28% by weight of the composition, said second gel is present in an amount between 71.5% and 77.5% by weight of the composition, the active principle is present in an amount between 0.2% and 5% by weight of the composition, the active principle is present in an amount between 0.2% and 5% by weight of the composition, and the anaesthetic is present in an amount between 1% and 5% by weight of the composition.

9. A composition according to claim 1 wherein the emulsifier agent in said first gel is triethanolamine.

10. A composition according to claim 1 wherein the preservative agent is a mixture of methyl parabene and propyl parabene.

11. A composition according to claim 1 additionally containing chlorhexidine and/or urea in a pharmaceutically effective amount.

12. A method for treating skin lesions comprising the application on the skin lesion of the person in need, a therapeutically effective amount of the composition according to claim 1.

13. The use of the composition according to claim 1 for treatment of local burns, grazes, erythema, eczema, herpes infection, avulsions, superficial sores and any skin damage leading to sloughing.

14. A composition according to claim 2 furthermore comprising an anaesthetic agent.

15. A composition according to claim 2 wherein said first thickener agent consists of carboxypolymethylene in an amount between 1.5 and 2.5% by weight of said first gel.

16. A composition according to claim 3 wherein said first thickener agent consists of carboxypolymethylene in an amount between 1.5 and 2.5% by weight of said first gel.

17. A composition according to claim 4 wherein said first thickener agent consists of carboxypolymethylene in an amount between 1.5 and 2.5% by weight of said first gel.

18. A composition according to claim 2 wherein said second thickener agent is carboxymethylcellulose in an amount between 1% and 4% by weight of said second gel.

19. A composition according to claim 3 wherein said second thickener agent is carboxymethylcellulose in an amount between 1% and 4% by weight of said second gel.

20. A composition according to claim 4 wherein said second thickener agent is carboxymethylcellulose in an amount between 1% and 4% by weight of said second gel.
